(19) United States
(12) Patent Application Publication
(10) Pub. No.: US 2025/0065137 A1
(43) Pub. Date: Feb. 27, 2025

(54) Garment Features for ECG Electrode Pressure and/or Stabilization in a Wearable Medical Device

(71) Applicant: ZOLL Medical Corporation, Chelmsford, MA (US)

(72) Inventors: Sunaina Rustagi, Presto, PA (US); Christopher Lawrence Swenglish, Connellsville, PA (US); Milad Alizadeh-Meghrazi, Etobicoke (CA); Tony Chahine, Etobicoke (CA); Ladan Eskandarian, Etobicoke (CA)

(21) Appl. No.: 18/717,372

(22) PCT Filed: Dec. 5, 2022
(86) PCT No.: PCT/US2022/051876

§ 371 Date: Jun. 6, 2024

Related U.S. Application Data
(60) Provisional application No. 63/286,466, filed on Dec. 6, 2021.

(51) Int. Cl. A61N 1/39

(52) U.S. Cl. CPC A61N 1/3904

(57) ABSTRACT

A non-invasive wearable ambulatory cardiac defibrillator configured to stabilize forces on one or more electrodes on the patient's skin is provided. The device includes a garment configured to be worn around a torso of a patient, a sensing electrode configured to sense electrical signals at the surface of the patient's skin indicative of electrical activity of the patient's heart, a therapy electrode configured to deliver defibrillation pulses to the patient, and a controller in communication with the sensing and therapy electrode. The controller is configured to receive the signals from the sensing electrode and to cause delivery of the defibrillation pulses from the therapy electrode based on the controller detecting a cardiac arrhythmia in the received electrical signals. The garment includes a main garment portion configured to engage the torso of the patient, an isolation zone material disposed within the main garment portion and to which the sensing electrode is attached, and a movement absorption region connecting the main garment portion to the isolation zone material.

### CROSS REFERENCE TO RELATED APPLICATION

This application claims priority to U.S. Provisional Patent Application No. 63/286,466, filed Dec. 6, 2021, the disclosure of which is hereby incorporated by reference in its entirety.

### FIELD OF THE DISCLOSURE

The present disclosure relates to wearable medical devices, and, more particularly, to a non-invasive wearable ambulatory cardiac defibrillator configured to improve sensing electrode contact with a patient's skin.

### BACKGROUND OF THE DISCLOSURE

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. As the wearable device is generally prescribed for continuous or near-continuous use (e.g., only to be removed when bathing), the patient wears the device during all daily activities such as walking, sitting, climbing stairs, resting or sleeping, and other similar daily activities. Maintaining continuous or near-continuous use of the device as prescribed can be important for monitoring patient progress as well as providing treatment to the patient if needed.

Wearable defibrillator garments include one or more electrodes for sensing electrical signals at the surface of the patient's skin. Such signals are indicative of electrical activity of the patient's heart, and can be monitored and analyzed to diagnose and treat arrhythmias. In order to effectively and reliably sense the electrical signals at the patient's skin, it is desirable that the sensing electrodes maintain consistent contact with the patient's skin.

### SUMMARY OF SOME OF THE EMBODIMENTS

Non-limiting examples of embodiments will now be described.

In an example, a non-invasive wearable ambulatory cardiac defibrillator is provided. The device comprises a garment configured to be worn around a torso of a patient; at least one sensing electrode attached to the garment and configured to sense electrical signal(s) at the surface of the patient's skin indicative of electrical activity of the patient's heart; at least one therapy electrode attached to the garment and configured to deliver one or more defibrillation pulses to the patient; and a controller in communication with the at least one sensing electrode and the therapy electrodes. The controller is configured to receive the electrical signal(s) from the at least one sensing electrode and to cause delivery of the one or more defibrillation pulses from the at least one therapy electrode based on the controller detecting a cardiac arrhythmia in the received electrical signal(s). The garment comprises: a main garment portion configured to engage the torso of the patient; an isolation zone material disposed within the main garment portion and to which one of the sensing electrodes is attached; and at least one movement absorption region connecting the main garment portion to the isolation zone material. The at least one movement absorption region has a different elasticity than the main garment portion and the isolation zone material.

The at least one movement absorption region can comprise a mesh having a less dense knit structure than at least one of the main garment portion and the isolation zone material.

The at least one movement absorption region can have a different thickness than at least one of the main garment portion and the isolation zone material.

The at least one movement absorption region can have a different stiffness than at least one of the main garment portion and the isolation zone material.

The garment can further comprise a plurality of connecting portions extending between the main garment portion and the isolation zone material.

The at least one movement absorption region can comprise a plurality of movement absorption regions arranged around a perimeter of the isolation zone material.

Each of the plurality of movement absorption regions can be arcuate.

The isolation zone material can be rectangular.

The isolation zone material can be circular.

The isolation zone material can comprise a reinforcing support film to which the one of the sensing electrodes is attached.

The at least one movement absorption region can be configured to induce a normal force on the one of the sensing electrodes ranging from about 0.1 psi to about 0.6 psi.

The at least one movement absorption region can be configured to induce a normal force on the one of the sensing electrodes ranging from about 0.3 psi to about 0.6 psi.

The at least one movement absorption region can be configured to induce a normal force on the one of the sensing electrodes ranging from about 0.4 psi to about 0.5 psi.

The garment can further comprise a plurality of isolation zones. Each of the at least one sensing electrode can be attached to one of the plurality of isolation zone materials.

The controller can be configured generate ECG information from the electrical signal(s) received from the at least one sensing electrode and to cause delivery of the one or more therapeutic pulses from the at least one therapy electrode.

In an example, a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; at least one therapy electrode attached to the garment and configured to deliver one or more defibrillation pulses to the patient; and a controller in communication with the at least one sensing electrode and the therapy electrodes. The controller is configured to receive the electrical signal(s) from the at least one sensing electrode and to cause delivery of the one or more defibrillation pulses from the at least one therapy electrode based on the controller detecting a cardiac arrhythmia in the received electrical signal(s). The garment comprises: a belt portion configured to wrap around the torso or waist of the patient; and at least one insert disposed in the belt portion. The at least one insert is configured to have a greater elasticity in a circumferential direction than an adjacent section of the belt portion.

The belt portion can be adjustable between a minimum circumference and a maximum circumference. A location of the at least one sensing electrode about a circumference of the belt portion can be different at the minimum circumference than at the maximum circumference due to stretching of the at least one insert.

A location of a first of the at least one electrodes changes by a first distance when the belt portion is adjusted from the minimum circumference to the maximum circumference. A location of a second of the at least one electrodes changes by a second distance when the belt portion is adjusted from the minimum circumference to the maximum circumference. The first distance can be different than the second distance.

The at least one insert can comprise a plurality of inserts.

Each of plurality of inserts can be disposed between two of the sensing electrodes to allow a distance between the two sensing electrodes to be adjusted by stretching the insert.

The plurality of inserts can be disposed asymmetrically about a circumference of the belt portion.

An elasticity of at least one of the plurality of the inserts can be different than an elasticity of at least one other of the plurality of inserts.

The controller can be configured generate ECG information from the electrical signal(s) received from the at least one sensing electrode and to cause delivery of the one or more therapeutic pulses from the at least one therapy electrode.

In an example, a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; at least one therapy electrode attached to the garment and configured to deliver one or more defibrillation pulses to the patient; and a controller in communication with the at least one sensing electrode and the therapy electrodes. The controller is configured to receive the electrical signal(s) from the at least one sensing electrode and to cause delivery of the one or more defibrillation pulses from the at least one therapy electrode based on the controller detecting a cardiac arrhythmia in the received electrical signal(s). The garment comprises at least one graduated thickness section to which the at least one sensing electrode is attached. The at least one graduated thickness section has a varying thickness. The at least one graduated thickness section is configured to apply a predetermined normal force to the at least one sensing electrode.

The at least one graduated thickness section can comprise a depression into which the at least one sensing electrode is at least partially recessed.

A depth of the depression can be less than a thickness of the at least one sensing electrode.

The varying thickness of the graduated thickness section can taper from a minimum thickness at an outer edge of the graduated thickness section to a maximum thickness at an inner region of the graduated thickness section.

The varying thickness of the graduated thickness section can taper non-linearly.

The graduated thickness section can comprise a constant thickness section where the at least one sensing electrode is attached to the graduated thickness section.

The at least one graduated thickness section can be configured to apply the predetermined normal force to the at least one sensing electrode ranging from about 0.1 psi to about 0.6 psi.

The at least one graduated thickness section can be configured to apply the predetermined normal force to the at least one sensing electrode ranging from about 0.3 psi to about 0.6 psi.

The at least one graduated thickness section can be configured to apply the predetermined normal force to the at least one sensing electrode ranging from about 0.4 psi to about 0.5 psi.

The controller is configured generate ECG information from the electrical signal(s) received from the at least one sensing electrode and to cause delivery of the one or more therapeutic pulses from the at least one therapy electrode.

In an example, a non-invasive wearable ambulatory cardiac defibrillator is provided. The device comprises a garment configured to be worn around a torso of a patient; at least one sensing electrode attached to the garment and configured to sense electrical signal(s) at the surface of the patient's skin indicative of electrical activity of the patient's heart; and a controller in communication with the at least one sensing electrode. The controller is configured to receive the electrical signal(s) from the at least one sensing electrode. The garment comprises: a main garment portion configured to engage the torso of the patient; an isolation zone material disposed within the main garment portion and to which one of the sensing electrodes is attached; and at least one movement absorption region connecting the main garment portion to the isolation zone material. The at least one movement absorption region has a different elasticity than the main garment portion and the isolation zone material.

In an example, a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; and a controller in communication with the at least one sensing electrode. The controller is configured to receive the electrical signal(s) from the at least one sensing electrode. The garment comprises: a belt portion configured to wrap around the torso or waist of the patient; and at least one insert disposed in the belt portion. The at least one insert is configured to have a greater elasticity in a circumferential direction than an adjacent section of the belt portion.

In an example, a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; and a controller in communication with the at least one sensing electrode. The controller is configured to receive the electrical signal(s) from the at least one sensing electrode. The garment comprises at least one graduated thickness section to which the at least one sensing electrode is attached. The at least one graduated thickness section has a varying thickness. The at least one graduated thickness section is configured to apply a predetermined normal force to the at least one sensing electrode.

### BRIEF DESCRIPTION OF THE DRAWINGS

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the disclosure.

Further features and other examples and advantages will become apparent from the following detailed description made with reference to the drawings.

FIG. 1A is detail view of an isolation zone material and movement absorption region of a non-invasive wearable ambulatory cardiac defibrillator, according to an example of the present disclosure;

FIG. 1B is detail view of an isolation zone material and movement absorption region of a non-invasive wearable ambulatory cardiac defibrillator, according to an example of the present disclosure;

FIG. 2 is detail view of an isolation zone material and movement absorption region of a non-invasive wearable ambulatory cardiac defibrillator, according to an example of the present disclosure;

FIG. 3A is detail view of the isolation zone material and movement absorption region of FIG. 2 in a first orientation;

FIG. 3B is detail view of the isolation zone material and movement absorption region of FIG. 2 in a second orientation;

FIG. 3C is detail view of the isolation zone material and movement absorption region of FIG. 2 in a third orientation;

FIG. 4 is a perspective view of a belt portion of a non-invasive wearable ambulatory cardiac defibrillator, according to an example of the present disclosure;

FIG. 5A is a perspective view of the belt portion of FIG. 4, shown in a minimum circumference state;

FIG. 5B is a perspective view of the belt portion of FIG. 4, shown in a maximum circumference state;

FIG. 6 is a schematic diagram of the belt portion of FIG. 4, shown with the minimum and maximum circumference states overlaid;

FIG. 7 is detail view of a graduated thickness section of a non-invasive wearable ambulatory cardiac defibrillator, according to an example of the present disclosure;

FIG. 8 is detail view of a graduated thickness section of a non-invasive wearable ambulatory cardiac defibrillator, according to an example of the present disclosure;

FIG. 9 is a force diagram of the isolation zone material and movement absorption region of any of FIGS. 1A, 2 and/or 3A-3C;

FIG. 10 is a schematic of an exemplary wearable cardiac monitoring and therapeutic medical device that can be used in connection with the present disclosure;

FIG. 11 is a front view of an exemplary support garment for the wearable cardiac monitoring and therapeutic medical device of FIG. 10 as worn on a patient;

FIG. 12 is a rear view of the support garment of FIG. 10 as worn on a patient;

FIGS. 13A and 13B are a front view of an exemplary support garment and electrode assembly, respectively, for the wearable monitoring and therapeutic medical device that can be used in connection with the present disclosure;

FIG. 14 is a schematic of an exemplary wearable cardiac monitoring and therapeutic medical device that can be used in connection with the present disclosure;

FIG. 15A is a schematic drawing showing a front perspective view of an example monitor for the wearable medical device of FIG. 14;

FIG. 15B is a schematic drawing showing a rear perspective view of the example monitor of FIG. 15A;

FIG. 16 is a schematic diagram of functional components of the wearable medical device of FIG. 14;

FIG. 17A is a bottom view of a therapy electrode of the medical device of FIG. 10; and

FIG. 17B is a top view of the therapy electrode of FIG. 17A.

### DETAILED DESCRIPTION OF SOME OF THE EMBODIMENTS

As used herein, the singular forms of “a”, “an”, and “the” include plural referents unless the context clearly dictates otherwise.

As used herein, the terms “right”, “left”, “top”, and derivatives thereof shall relate to the disclosure as it is oriented in the drawing figures. However, it is to be understood that the disclosure can assume various alternative orientations and, accordingly, such terms are not to be considered as limiting. Also, it is to be understood that the disclosure can assume various alternative variations and stage sequences, except where expressly specified to the contrary. It is also to be understood that the specific devices and processes illustrated in the attached drawings and described in the following specification are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about” or “approximately”. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Also, it should be understood that any numerical range recited herein is intended to include all sub-ranges subsumed therein. For example, a range of “1 to 10” is intended to include any and all sub-ranges between and including the recited minimum value of 1 and the recited maximum value of 10; that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

Patients who are at risk, have been hospitalized for, or otherwise are suffering from, adverse heart conditions can be prescribed a wearable cardiac monitoring and/or treatment device. As the wearable device is generally prescribed for continuous or near-continuous use (e.g., only to be removed when bathing), the patient wears the device during all daily activities such as walking, sitting, climbing stairs, resting or sleeping, and other similar daily activities. Maintaining continuous or near-continuous use of the device as prescribed can be important for monitoring patient progress as well as providing treatment to the patient if needed.

In order to effectively and reliably sense the electrical signals at the patient's skin, the disclosure herein relates to techniques and systems for enabling electrodes to maintain consistent contact with the patient at an appropriate pressure range and with minimal lifting and/or shifting of the electrodes on the patient's skin. For example, wearable garments are secured to patients such that the garments rely on tension in the garment fabric to hold sensing electrode(s) against the patient's skin. The inherent flexibility of such wearable defibrillator garments can result in the ECG sensing electrodes shifting on the patient or separating from skin contact, affecting signal quality. Features are described herein to ensure that sensing electrodes attached to the garments maintain consistent contact with the patient's skin at an appropriate pressure range and minimize shifting and/or lifting of such sensing electrodes.

In examples described herein, relative fabric stiffness and/or elasticity properties can be measured in the following ways. In one example, stiffness (and/or fabric elasticity) can be measured, tested and/or recorded on a Universal Testing Machine or an INSTRON-4411 tensile test machine (CRE type) from INSTRON of Norwood, MA. In this example, the stiffness of two samples, one representing a first portion or component and the other representing a second portion or component can be characterized as two separate graphs of force (e.g., in pounds per inch) over distance. The slopes of the two graphs can be used to determine relative stiffness of the two sample materials. In examples, testing of the samples can be performed in accordance with the procedures described in ASTM D 4964, Standard Test Method for Tension and Elongation of Elastic Fabrics (Constant-Rate-of-Extension Type Tensile Testing Machine). The fabric materials can be tested in accordance with other relevant testing standards, such as, ASTM D 5278, Standard Test Method for Elongation of Narrow Elastic Fabrics (Static-Load Testing), and ASTM D2731-21, Standard Test Method for Elastic Properties of Elastomeric Yarns (CRE Type Tensile Testing Machines). In another example, stretch and/or elasticity of a fabric can be measured in terms of a Poisson's Ratio, which is deformation of the specimen fabric material in directions perpendicular to the specific direction of loading. In this example, the ratio is characterized in terms of transverse strain over axial strain. In another example, stretch and/or elasticity of a fabric material can be measured in terms of “stretch and recovery,” where the specimen fabric material is stretched to a maximum limit without being deformed and such is measured in terms of a percentage per unit length. For example, such stretch can be along one axis or two orthogonal axes depending on the material and purpose of use of the material as described herein.

As summarized above, examples disclosed herein are directed to a non-invasive wearable ambulatory cardiac defibrillator configured to stabilize forces on one or more sensing electrodes on a patient's skin. These wearable medical devices are used in clinical or outpatient settings to monitor and/or record various electrocardiogram (ECG) and other physiological signals of a patient. In some examples, these wearable medical devices can analyze the ECG and other physiological signals to monitor for arrhythmias, and provide treatment such as cardioverting, defibrillating, or pacing shocks/pulses via therapy electrodes in the event of life-threatening arrhythmias. Examples of cardiac monitoring and treatment devices that can implement the adjustable garment features and/or processes described herein includes wearable defibrillators, which are also called wearable cardioverter defibrillator (WCDs); and hospital wearable defibrillators (HWDs).

The sensing electrodes as described herein are configured to sense electrical signals at the surface of the patient's skin. Such signals are indicative of electrical activity of the patient's heart and can be used to construct electrocardiogram (ECG) or other models used to monitor the patient's heart. In particular, a controller can receive and analyze the sensed electrical signals, and, in some examples, deliver treatment pulses/shocks via therapy electrodes. Ensuring that the sensing electrodes reliably and accurately detect the electrical signals from the patient's skin is therefore critical to the monitoring and therapy of at-risk patients.

Significant sources of erroneous and incomplete sensing of signals include shifting of the sensing electrodes on the patient's skin, separation of the sensing electrodes from the patient's skin, and inadequate pressure of the sensing electrode against the patient's skin. An acceptable contact pressure range at one or more electrode-to-skin interfaces can be selected based upon a minimum pressure that provides adequate contact between the sensing electrodes and the patient's skin to facilitate essentially complete transmission of the pertinent electrical signals from the patient to the electrodes. For example, an acceptable pressure range at one or more electrode-to-skin interfaces can include pressures, in some implementations, ranging from about 0.25 psi to about 0.62 psi or 0.25 psi to 0.62 psi, in some implementations, about 0.4 psi to about 0.62 psi or 0.4 psi to 0.62 psi, or in some implementations about 0.5 psi to about 0.62 psi or 0.5 psi to 0.62 psi.

The devices described herein can include features to provide an acceptable range of pressure (e.g., about 0.25 psi to about 0.62 psi), via a substantially uniform normal force acting substantially perpendicular to the patient's skin, to the sensing electrodes. This substantially uniform normal force can prevent displacement of the sensing electrodes and counteract separation of the electrode-to-skin interface so that the sensing electrodes can reliably sense electrical signal(s) at the patient's skin.

In examples, the devices described herein can include a wearable garment having an isolation zone material to which one of the sensing electrodes is attached. The isolation zone material is connected to the garment by a movement absorption region. The movement absorption region has a different elasticity than the main portion of the garment, allowing the isolation zone material to exert a consistent pressure within an acceptable range (e.g., about 0.25 psi to about 0.62 psi, as described herein) to the sensing electrodes. Thus, a consistent, reliable electrical interface between the sensing electrode and the patient's skin is established, improving the efficacy of patient monitoring and therapy. In some examples, the garment can further include one or more connecting portions extending over the movement absorption region and connecting to the isolation zone material directly to the main portion of the garment, which can improve stability of the electrode relative the patient's skin.

In examples, the garment can include a belt portion having an adjustable circumference in order to accommodate patients of different sizes. To position the electrodes attached to the belt portion at the most clinically desirable locations on the patient, the belt portion includes at least one insert allowing the belt portion to stretch and thereby adjust the distance between the sensing electrodes. As such, the distance between sensing electrodes varies proportionally with the circumference of the belt portion as the circumference of the belt portion is adjusted. The inserts can have a greater elasticity in a circumferential direction than an adjacent section of the belt portion to allow the aforementioned stretching.

In examples, the devices described herein can include a wearable garment having a graduated thickness portion to which one of the sensing electrodes is attached. The graduated thickness portion has a varying thickness and is configured to apply a predetermined normal force to the sensing electrode, thereby maintaining a consistent pressure within an acceptable range (e.g., about 0.25 psi to about 0.62 psi, as described herein) on the sensing electrodes. Thus, a consistent, reliable electrical interface between the sensing electrode and the patient's skin is established, improving the efficacy of patient monitoring and therapy.

While the examples described herein are generally directed to cardiac defibrillator devices, the present disclosure also encompasses cardiac cardioversion devices and cardiac monitoring devices. Thus, the examples described herein need not includes therapy components such as therapy electrodes, and instead can be strictly used for patient monitoring.

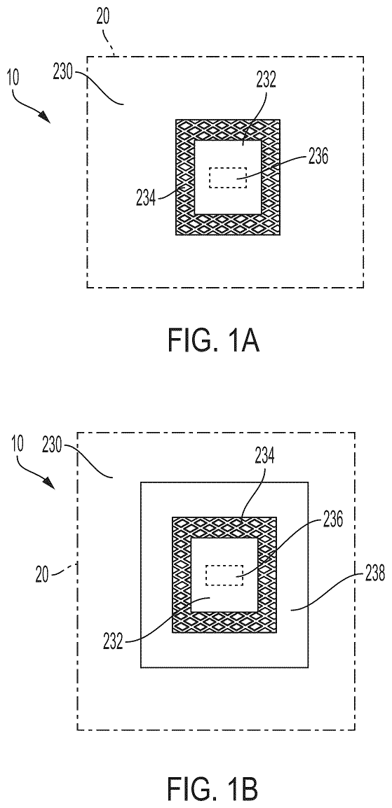

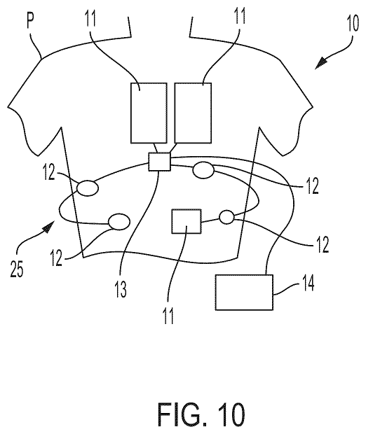

Referring now to the accompany drawings, FIGS. 1A-8 illustrate examples of non-invasive wearable ambulatory cardiac defibrillators 10 according to the present disclosure. In the various examples shown in FIGS. 1A-8, components with like reference numerals can refer to like part from other examples. Each example of the device 10 generally includes at least one sensing electrode 12 configured to sense electrical signal(s) at the surface of the patient's skin indicative of electrical activity of the patient's heart. The sensed electrical signal(s) can subsequently be used by additional components of the device 10 (or a third-party device) to monitor the patient and/or provide therapy if deemed necessary. The sensing electrodes 12 are attached to a support garment 20 configured to be worn around the torso of the patient P (as shown, e.g., in FIG. 10). The support garment 20 holds the sensing electrodes 12 at a clinically advantageous location with respect to the torso of the patient P. Importantly, the garment 20 retains the sensing electrodes 12 in position and prevents the sensing electrodes 12 from shifting or separating from the patient's skin, thereby ensuring that the electrical signal(s) are reliably received by the sensing electrodes 12. In addition, the garment 20 can include features to provide a consistent pressure within an acceptable range (e.g., about 0.25 psi to about 0.62 psi, as described herein) on the sensing electrodes 12. Further details of the device 10, including the garment 20 and other components thereof, and associated systems are shown and described herein with reference to FIGS. 10-17B.

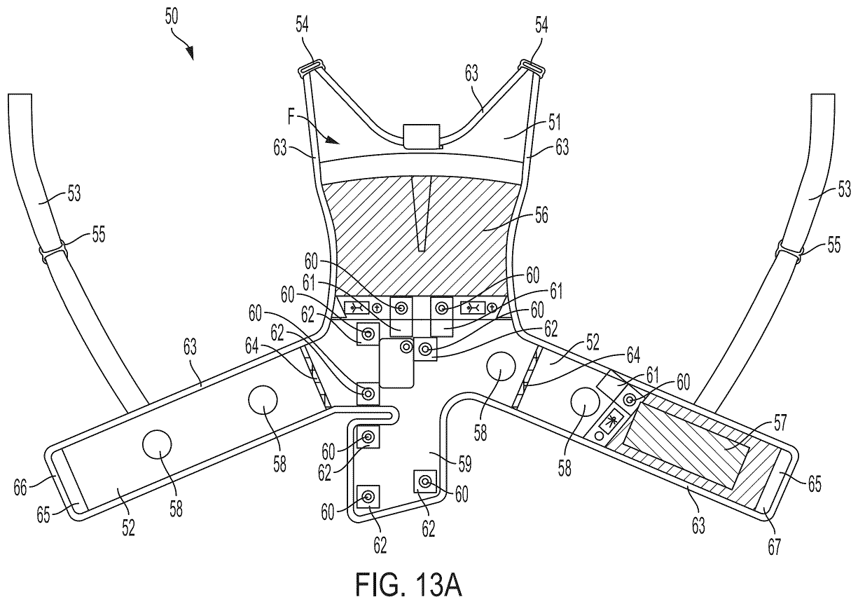

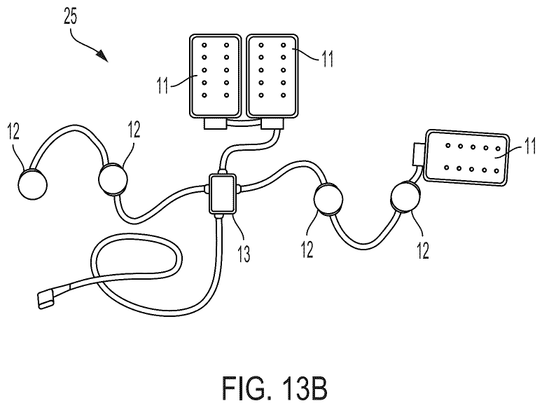

Referring now to the example shown in FIG. 1A, the garment 20 can include a main garment portion 230 configured to engage the skin of the torso of the patient P. In some examples, the main garment portion 230 can make up a majority of an internal surface of the garment 20. In some examples, the main garment portion 230 can be made of an elastic, low spring rate material composition based on a fiber content of about 20% elastic fiber, about 32% polyester fiber, and up to about 48% or more of nylon or other fiber. The garment 20 can further include one or more isolation zone materials 232 to which the sensing electrodes 12 are mounted. Each isolation zone material 232 can include, for example, one of the attachment points 58 described herein with reference to FIGS. 13A-13B. In some examples, each isolation zone material 232 can include a reinforcing support film 236 to which the associated sensing electrode 12 is mounted. The reinforcing support film 236 can include, in some examples, a hook-and-loop fastener patch configured to connect to a corresponding hook-and-loop fastener patch on the associated sensing electrode 12. In some examples, each isolation zone material 232 can be rectangular or circular in shape, though other shapes are within the scope of the present disclosure.

Each isolation zone material 232 can be at least partially surrounded by a movement absorption region 234 which connects the isolation zone materials 232 to the main garment portion 230. In some examples, each movement absorption region 234 can be rectangular or circular in shape, though other shapes are within the scope of the present disclosure. Each movement absorption region 234 can be made of an elastic, fabric material containing elastic fiber, nylon fiber, polyester fiber, and/or other suitable fibers. The movement absorption region 234 allows the associated isolation zone material 232 to remain substantially stable relative to the skin of the patient P even as the main garment portion 230 shifts with patient movement. Thus, the isolation zone material 232 is able to maintain a consistent pressure within an acceptable range (e.g., about 0.25 psi to about 0.62 psi, as described herein) on sensing electrode 12. As shown in FIG. 19, the movement absorption region 234 is configured to stretch such that tension within the movement absorption region 234 causes the associated isolation zone material 232 to bear against the associated sensing electrode 12 at a pressure within the acceptable range (e.g., about 0.25 psi to about 0.62 psi, as described herein). In some examples, each movement absorption region is configured to induce a normal force Non the sensing electrode 12 ranging from about 0.1 psi to about 0.6 psi, or from 0.1 psi to 0.6 psi. In some examples, each movement absorption region 234 is configured to induce a normal force N on the sensing electrode 12 ranging from about 0.3 psi to about 0.6 psi, or from 0.3 psi to 6 psi. In some examples, each movement absorption region 234 is configured to induce a normal force N on the sensing electrode 12 ranging from about 0.4 psi to about 0.5 psi, or from 0.4 psi to 0.5 psi.

In some examples, the movement absorption regions 234 can have a different elasticity that the main garment portion 230 in order to achieve the desired behavior of the movement absorption regions 234. In particular, the movement absorption regions 234 can be more clastic (i.e. less stiff) than the main garment portion 230 and/or the isolation zone material 232 so that the movement absorption region 234 can stretch to maintain a consistent pressure on the associated sensing electrode 12. The stiffness of the movement absorption regions 234 relative to the main garment portion 230 and/or the isolation zone material 232 can be defined in accordance with any applicable ASTM standard described herein. In some examples, the relatively greater elasticity of the movement absorption region 234 can be achieved by making the movement absorption region 234 from a mesh material having a less dense knit structure than the main garment portion 230 and/or the isolation zone material 232. In some examples, the relatively greater elasticity of the movement absorption region 234 can be achieved by making the movement absorption region 234 from a material having a lower stiffness (more elasticity) than the main garment portion 230 and/or the isolation zone material 232. In implementations, such stiffness and/or elasticity can characterized in terms of a ratio relating the movement absorption region 234 to the main garment portion 230 and/or the isolation zone material 232 as follows. In some examples, the relatively greater elasticity of the movement absorption region 234 can be achieved by making the movement absorption region 234 from a material having a lower stiffness than the main garment portion 230 and/or the isolation zone material 232. For example, the stiffness of the movement absorption region 234 can be about 40% to about 50% less than the stiffness of the main garment portion 230 and/or the isolation zone material 232. In another example, the stiffness of the movement absorption region 234 can be about 50% to about 60% less than the stiffness of the main garment portion 230 and/or the isolation zone material 232. In another example, the stiffness of the movement absorption region 234 can be about 60% to about 75% less than the stiffness of the main garment portion 230 and/or the isolation zone material 232. In another example, the stiffness of the movement absorption region 234 can be about 75% to about 95% less than the stiffness of the main garment portion 230 and/or the isolation zone material 232. Alternatively, in other examples, the movement absorption regions 234 can be less elastic (i.e. more stiff) that the main garment portion 230 and/or the isolation zone material 232.

In order to establish reliable interfaces with the sensing electrodes 12, the isolation zone materials 232 can be more stiff than the main garment portion 230, as defined by any of the applicable ASTM standards described herein. For example, the stiffness of the isolation zone material 232 can be about 40% to about 50% more than the stiffness of the main garment portion 230. In another example, the stiffness of the isolation zone material 232 can be about 50% to about 60% more than the stiffness of the main garment portion 230. In another example, the stiffness of the isolation zone material 232 can be about 60% to about 75% more than the stiffness of the main garment portion 230. In another example, the stiffness of the isolation zone material 232 can be about 75% to about 95% more than the stiffness of the main garment portion 230. In some examples, the isolation zone materials 232 can have a denser knit structure than the main garment portion 230.

Referring now to FIG. 1B, another example of the garment 20 includes an intermediate region 238 disposed between the main garment portion 230 and the movement absorption region 234. The intermediate region can provide an additional level of flexibility/stretchability to achieve consistent pressure within the acceptable pressure range (e.g., about 0.25 psi to about 0.62 psi, as described herein) on the associated sensing electrode 12. The intermediate region 238 can have a different elasticity than the main garment portion 230 and/or the movement absorption region 234, as defined by any of the applicable ASTM standards described herein. In some examples, the intermediate region 238 can have a coarser knit structure than the main garment portion 230 and/or the movement absorption region 234. In some examples, the intermediate region 238 can have a denser knit structure than the main garment portion 230 and/or the movement absorption region 234.

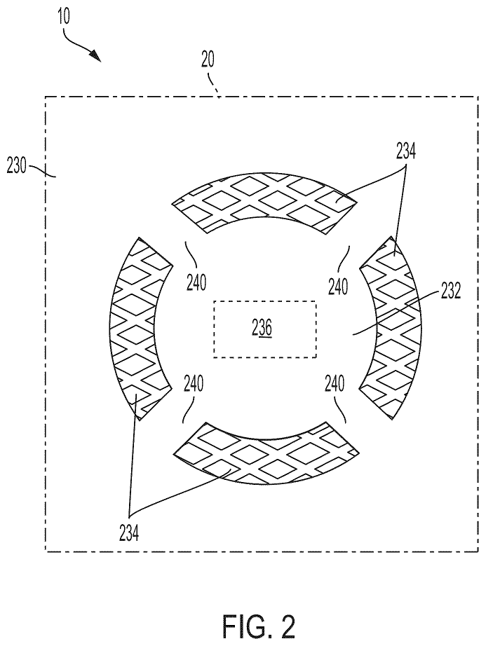

Referring now to FIG. 2, in some examples, the garment 20 further includes a plurality of connecting portions 240 extending between the main garment portion 230 and the isolation zone material 232. The plurality of connecting portions 240 extend across or interrupt the movement absorption region 234, effectively dividing the movement absorption region into 234 into a plurality of movement absorption regions 234 arranged around a perimeter of the isolation zone material 232. Each of the plurality of movement absorption regions 234 can be arcuate in shape as shown in FIG. 2, though other shapes are within the scope of the present disclosure. In some examples, the plurality of the connecting portions 240 can be made of the same material as the main garment portion 230 and/or the isolation zone material 232. In some examples, the main garment portion 230, the plurality of the connecting portions 240, and isolation zone material 232 can be constructed of a continuous material section. The presence of the plurality of connecting portion 240 can, in some examples, improve stabilization of the isolation zone material 232 and the associated sensing electrode 12.

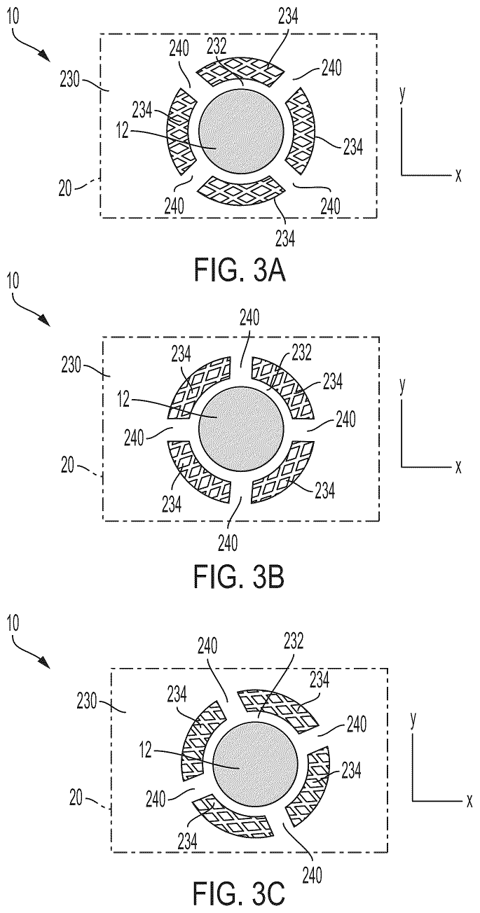

FIGS. 3A-3C illustrate the same example as FIG. 2, except that the plurality of movement absorption regions 234 are rotated to various orientation various orientations relative to an x-y plane. Additionally, the sensing electrodes 12 are shown attached to the isolation zone materials 232 in FIGS. 3A-3C. The x- and y-axes shown in FIGS. 3A-3C represent horizontal and vertical directions, respectively, with the garment in position of the patient's torso. In FIG. 3A, the connecting portions 240 are oriented at about 45° angles relative to the x- and y-axes. In FIG. 3B, two of the connecting portions 240 are substantially parallel to the x-axis, and two of the connecting portions 240 are substantially parallel to the y-axis. In FIG. 3C, the connecting portions 240 are rotated to an orientation between the orientations of FIGS. 3A and 3B. The various rotational orientations of the connecting portions 240 shown in FIGS. 3A-3C can improve stability of the isolation zone materials 232 and the associated sensing electrodes 12 in different directions in the x-y plane. Similarly, the mesh structure of the plurality of movement absorption regions 234 can be rotated to improve stability of the isolation zone materials 232 and the associated sensing electrodes 12 in different directions in the x-y plane. Thus, the connecting portions 240 associated with different sensing electrodes 12 of the device 10 can be oriented in different positions with respect to the x-y plane depending on anticipated external forces at the electrode 12 locations.

In examples of the device 10, any or all of the sensing electrodes 12 can be attached to an isolation zone material 232 surrounded by a movement absorption region 234 as described in any of the examples of FIGS. 1A-3C. In some examples of the device 10, an isolation zone material 232 surrounded by a movement absorption region 234 is only provided for certain sensing electrodes 12 configured to engage portions of the patient P that experience relatively high levels of motion, while other sensing electrodes 12 are attached directly to the main garment portion 230. In some examples, one of the sensing electrodes 12 of the device 10 can be attached to an isolation zone material 232 surrounded by a movement absorption region 234 as described in one of the examples of FIGS. 1A-3C, and another of the sensing electrodes 12 of the device 10 can be attached to an isolation zone material 232 surrounded by a movement absorption region 234 as described in another of the examples of FIGS. 1A-3C. Thus, combinations of the various examples of the isolation zone materials 232 and movement absorption regions 234 provided in FIGS. 1A-3C are possible within the same device 10.

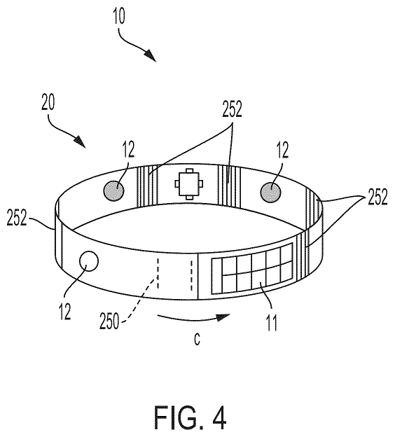

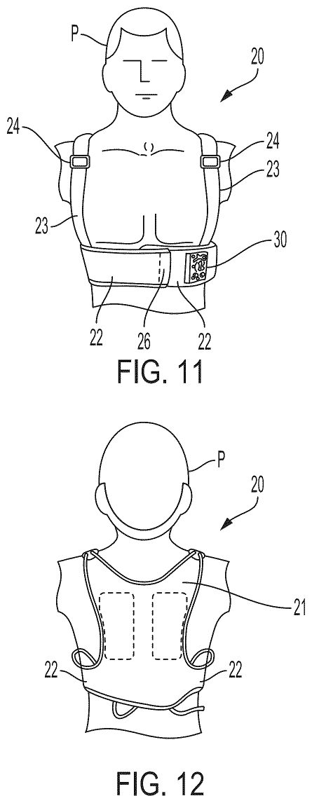

Referring now to FIGS. 4-6, a belt portion 250, which can correspond to the belt 22 of FIGS. 11-12 or to the belt 52 of FIG. 13A, of the garment 20 can have an adjustable circumference to accommodate patients of different sizes. The belt portion 250 can include at least one insert 252 having a greater elasticity in a circumferential direction C than an adjacent section of the belt portion 250. Each of the inserts 252 can be arranged between two of the sensing electrodes 12 such that a distance between the two sensing electrodes 12 can be adjusted by stretching the insert 252. That is, each insert 252 can expand to increase a distance between sensing electrodes 12 on either side of the insert 252, and each insert 252 can relax to decrease the distance between sensing electrodes 12 on either side of the insert 152. The expansion and relaxation of the insert 252, and the corresponding distance change between the sensing electrodes 12 on either side of the insert 252, allows the distance between the sensing electrodes 12 to vary proportionally with the circumference of the belt portion 250 as the circumference of the belt portion 250 is adjusted. Thus, the sensing electrodes 12 are caused to align with clinically desirable locations on the patient's torso regardless of the circumference to which the belt portion 250 is set based on the size of the patient P.

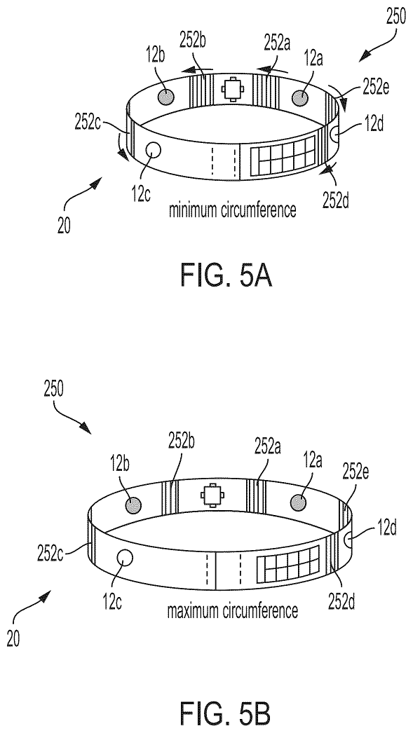

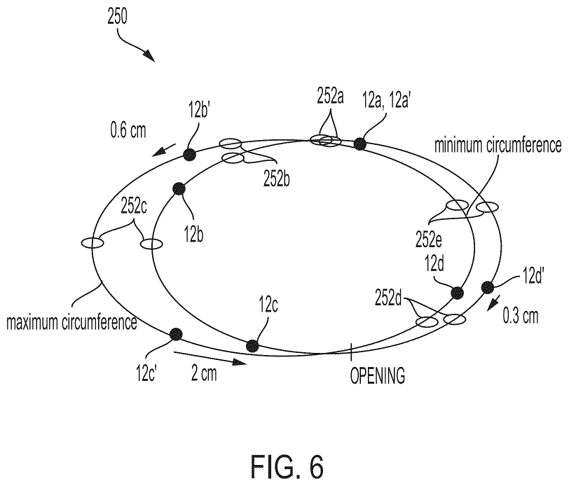

With specific reference to FIGS. 5A-6, the belt portion 250 can be adjustable between a minimum circumference size and a maximum circumference size to accommodate patients P of different waist size. The minimum circumference size, shown in FIG. 5A, can correspond to the circumference of the belt portion 250 when adjusted for a patient P having the minimum waist size the garment 20 can accommodate. The maximum circumference, shown in FIG. 5B size, can correspond to the circumference of the belt portion 250 when adjusted for a patient P having the maximum waist size for which the garment 20 can accommodate. In the example shown in FIGS. 5A, 5B, and 12, the belt portion 250 has four sensing electrodes 12a-12d attached thereto, and five inserts 252a-252e interspersed with the sensing electrodes 12a-12d.

FIG. 6 schematically shows the belt portion 250 in the minimum circumference state overlaid with the belt portion 250 in the maximum circumference state. Using the position of the first sensing electrode 12a as a reference position which does not move, FIG. 6 shows the change in position of the other sensing electrodes 12b-12d as the inserts 252 stretch from the minimum circumference state to the maximum circumference state. The second sensing electrode 12b on the minimum circumference moves circumferentially 0.6 centimeters (cm) to the position of the second sensing electrode 12b′ on the maximum circumference. The third sensing electrode 12c on the minimum circumference moves circumferentially 2 cm to the position of the third sensing electrode 12c′ on the maximum circumference. The fourth sensing electrode 12d on the minimum circumference moves circumferentially 0.3 cm to the position of the fourth sensing electrode 12d′ on the maximum circumference. As such, the positions of the four sensing electrodes 12a-12d can be aligned with desired locations on the patient's torso whether the belt portion 250 is used on a relatively small patient P in the minimum circumference state, or on a relatively large patient P in the maximum circumference state. Similarly, the belt portion 250 can be adjusted to any state between the minimum circumference and maximum circumference, and the sensing electrodes 12a-12d will align with desired locations on a correspondingly-sized patient's torso.

As can be appreciated from FIG. 6, the inserts 252 can be designed and arranged such that different sensing electrodes 12 move different distances as the belt portion 250 is adjusted. In some examples, the inserts 252 can be arranged symmetrically around the circumference of the belt portion 250 to achieve the desired positional movement of the sensing electrodes 12. In other examples, the inserts 252 can be arranged asymmetrically around the circumference of the belt portion 250 to achieve the desired positional movement of the sensing electrodes 12. In some examples, the number of inserts 252 can be greater than, less than, or equal to the number of sensing electrodes attached to the belt portion 250. In some examples, more than one insert 252 can be disposed between a pair of adjacent sensing electrodes 12.

As noted herein, the inserts 252 can have a different elasticity that adjacent sections of the belt portion 250. The difference in elasticity between the inserts 252 and the adjacent section of the belt portion 250 can be defined by any of the applicable ASTM standards described herein. For example, the stiffness of the adjacent section of the belt portion 250 can be around 40%-50% more than the stiffness of the inserts 252. In another example, the stiffness of the adjacent section of the belt portion 250 can be about 50% to about 60% more than the stiffness of the inserts 252. In another example, the stiffness of the adjacent section of the belt portion 250 can be about 60% to about 75% more than the stiffness of the inserts 252. In another example, the stiffness of the adjacent section of the belt portion 250 can be about 75% to about 95% more than the stiffness of the inserts 252. In addition, some of the inserts 252 can have greater elasticity than other of the inserts 252, such that certain sections of the belt portion 250 can stretch farther in the circumferential direction C than other sections of the belt portion 250. Selecting an appropriate size and/or elasticity for each of the inserts 252 allows for control of the distance that each sensing electrode 12 moves when the belt portion 250 is transitioned from the minimum circumference to het maximum circumference, and vice versa.

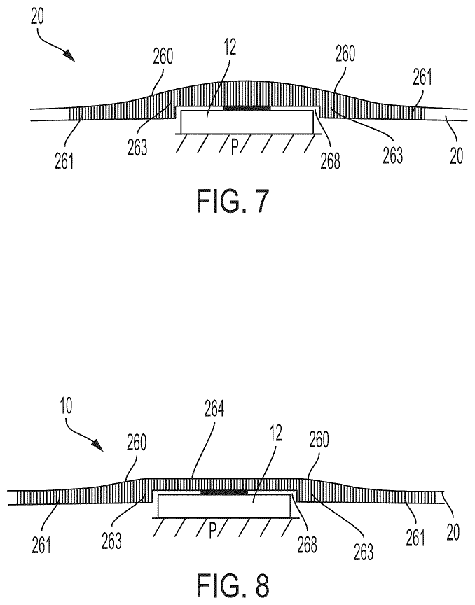

Referring now to FIGS. 7 and 8, in some examples, the garment 20 can include at least one graduated thickness section 260 to which the sensing electrodes 12 are attached. Each graduated thickness section 260 is configured to apply a predetermined normal force to the attached sensing electrode 12 to ensure reliable electrical connection between the sensing electrode 12 and the patient's skin. The normal force can particularly induce a pressure on the sensing electrode 12 within the acceptable range (e.g., about 0.25 psi to about 0.62 psi, as described herein). In some examples, the graduated thickness section 260 can be configured to induce a normal force on the sensing electrode 12 ranging from about 0.1 psi to about 0.6 psi. In some examples, the graduated thickness section 260 can be configured to induce a normal force on the sensing electrode 12 ranging from about 0.3 psi to about 0.6 psi, or from 0.3 psi to 0.6 psi. In some examples, the graduated thickness section 260 can be configured to induce a normal force on the sensing electrode 12 ranging from about 0.4 psi to about 0.5 psi, or from 0.4 psi to 0.5 psi.

Each graduated thickness section 260 can have a varying thickness tapering from a minimum thickness tmin at an outer edge 261 of the graduated thickness section 260 to a maximum thickness tmax at an inner region 263 of the graduated thickness section 260. The varying thickness of the graduated thickness section 260 can taper linearly or non-linearly. The specifications of the graduated thickness section 260, including the minimum thickness tmin, maximum thickness tmax, and profile of the taper can be selected to provide the predetermined normal force, and consequently the desired pressure (e.g., about 0.25 psi to about 0.62 psi, as described herein), to the sensing electrode 12. In some examples, as shown in FIG. 8, the graduated thickness section 260 includes a constant thickness section 264 where the associated sensing electrode 12 is attached to the graduated thickness section 260.

In some examples, the graduated thickness section 260 can include a depression 268 into which the attached sensing electrode 12 is at least partially recessed. The depth of the depression 268 is less than a thickness of the sensing electrode 12 so that the sensing electrode protrudes from the depression 268 in order to contact the patient's skin.

In some examples, the graduated thickness section 260 can be continuously formed with the material forming the remainder in the garment 20. In some examples, the graduated thickness section 260 can be a separate piece of material adhered, bonded, or otherwise connected to the garment 20.

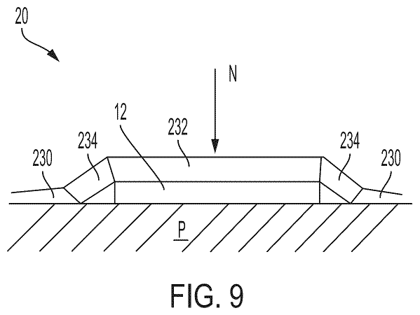

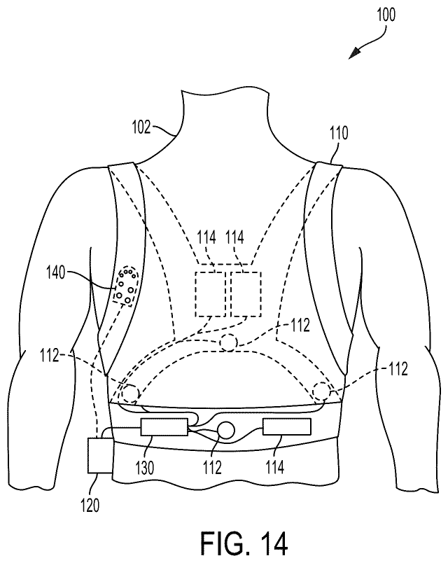

Having described various examples of features for improving the interface between the sensing electrodes 12 of the wearable medical device 10 and the patient, additional explanation of the wearable medical device 10 and associated systems will now be provided. FIG. 10 illustrates an exemplary wearable medical device 10, such as a wearable defibrillator, that is external, non-invasive, ambulatory, and wearable by a patient P and is configured to implement one or more configurations described herein. For example, the wearable medical device 10 can correspond to and/or include features of the examples shown in FIGS. 1A-9. The wearable medical device 10 can be an external or non-invasive medical device, e.g., the device 10 configured to be located substantially external to the patient P. The wearable defibrillator 10 can be worn or carried by an ambulatory patient P. According to one example of the present disclosure, the wearable defibrillator 10 is used as an ambulatory cardiac monitoring and treatment device within a monitoring and treatment system according to the present disclosure. FIGS. 14-16, discussed in detail below, illustrate in further detail an exemplary wearable medical device 100 in accordance with the present disclosure.

In accordance with one or more examples, a support garment 20 incorporating the features described herein is provided to keep the electrodes 11 and sensing electrodes 12 in place against the patient's body while remaining comfortable during wear. FIGS. 11 and 12 illustrate such a support garment 20 in accordance with an example of the present disclosure.

In order to obtain a reliable ECG signal so that the monitor can function effectively and reliably, the sensing electrodes 12 must be in the proper position and in good contact with the patient's skin. The electrodes 12 need to remain in a substantially fixed position and not move excessively or lift off the skin's surface. If there is excessive movement or lifting, the ECG signal will be adversely affected with noise and can cause problems with the arrhythmia detection and in the ECG analysis and monitoring system. Similarly, in order to effectively deliver the defibrillating energy, the therapy electrodes 11 are configured to remain in position and in contact with the patient's skin.

In accordance with one or more examples, the support garment 20 as described in this disclosure can provide comfort and functionality under circumstances of human body dynamics, such as bending, twisting, rotation of the upper thorax, semi-reclining, and lying down. These are also positions that a patient can assume if he/she were to become unconscious due to an arrhythmic episode. The design of the garment 20 is generally such that it minimizes bulk, weight, and undesired concentrations of force or pressure while providing the necessary radial forces upon the treatment and sensing electrodes 11, 12 to ensure device functionality. A wearable defibrillator monitor 14 can be disposed in a support holster operatively connected to or separate from the support garment 20. The support holster can be incorporated in a band or belt worn about the patient's waist or thigh.

As shown in FIGS. 11 and 12, in some implementations, the support garment 20 as described in this disclosure is provided in the form of a vest or harness having a back portion 21 and sides extending around the front of the patient P to form a belt 22. The ends of the belt 22 are connected at the front of the patient P by a closure 26, which can comprise one or more clasps. Multiple corresponding closures can be provided along the length of the belt 22 to allow for adjustment in the size of the secured belt 22 in order to provide a more customized fit to the patient P. The support garment 20 can further include two straps 23 connecting the back portion 21 to the belt 22 at the front of the patient P. The straps 23 have an adjustable size to provide a more customized fit to the patient P. The straps 23 can be provided with sliders 24 to allow for the size adjustment of the straps 23. The straps 23 can also be selectively attached to the belt 22 at the front of the patient P. The support garment 20 can be comprised of an elastic, low spring rate material that stretches appropriately to keep the electrodes 11, 12 in place against the patient's skin while the patient P moves and is lightweight and breathable. For example, the support garment 20 can have elastic, low spring rate material composition based on a fiber content of about 20% clastic fiber, about 32% polyester fiber, and up to about 48% or more of nylon or other fiber.

In accordance with one or more examples, the support garment 20 as described in this disclosure is formed from an clastic, low spring rate material and constructed using tolerances that are considerably closer than those customarily used in garments. The materials for construction are chosen for functionality, comfort, and biocompatibility. The materials can be configured to wick perspiration from the skin. The support garment 20 can be formed from one or more blends of nylon, polyester, and spandex fabric material. Different portions or components of the support garment 20 can be formed from different material blends depending on the desired flexibility and stretchability of the support garment 20 and/or its specific portions or components. For instance, the belt 22 of the support garment 20 can be formed to be more stretchable than the back portion 21. According to one example, the support garment 20 as described in this disclosure is formed from a blend of nylon and spandex materials, such as a blend of about 77% nylon and about 23% spandex. According to another example, the support garment 20 as described in this disclosure is formed from a blend of nylon, polyester, and spandex materials, such as about 40% nylon, about 32% polyester, and about 14% spandex. According to another example, the support garment 20 as described in this disclosure is formed from a blend of polyester and spandex materials, such as about 86% polyester and about 14% spandex or about 80% polyester and about 20% spandex. For example, the nylon and spandex material is configured to be aesthetically appealing, and comfortable, e.g., when in contact with the patient's skin. Stitching within the support garment 20 can be made with industrial stitching thread. According to one example, the stitching within the support garment 20 is formed from a cotton-wrapped polyester core thread.

FIGS. 13A and 13B illustrate an exemplary support garment 50 according to the present disclosure. The support garment 50 incorporates additional improvements for enhancing the patient's experience in wearing the support garment for an extended period of time. The support garment examples provided herein promote comfort, aesthetic appearance, and case of use or application for older patients, or patients with physical infirmities and/or who are physically challenged, including patients with rheumatic conditions, patients with arthritis, and/or patients with autoimmune or inflammatory diseases that affect joints, tendons, ligaments, bones, and muscles of the arm and hand. Patients afflicted with such conditions can properly and/or correctly don the garments described herein. Features of the support garments can also help minimize the time needed by patients to assemble, don or remove the support garment. Further, patients benefit from such features, which can facilitate longer wear times, better patient compliance, and improve the reliability of the detected physiological signals and treatment of the patient. These features promote case of use, comfort and an aesthetic appearance for such patient populations. For example, the garments described herein generally follow design principles as noted below (e.g., similar to those prescribed in the Arthritis Foundation Guidelines).
Removing, donning, and assembling the garment and associated components do not require fine motor control or simultaneous actions,
Replacing electrodes and other components is possible for patients with limited reach and strength,
The garment and/or components include surface and/or textural aspects that makes the garment and/or components easy to grip and control.
The garment and/or components include features designed to minimize simultaneous actions such as depressing and pulling,
The garment and/or components include features to provide positive feedback (for example, “snap”, “click”, among others).

These features can encourage patients to wear the support garment and associated medical device for longer and/or continuous periods of time with minimal interruptions in the periods of wear. For example, by minimizing interruptions in periods of wear and/or promoting longer wear durations, patients and caregivers can be assured that the device is providing desirable information about as well as protection from adverse cardiac events such as ventricular tachycardia and/or ventricular fibrillation, among others. Moreover, when the patient's wear time and/or compliance is improved, the device can collect information on arrhythmias that are not immediately life-threatening, but can be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours. The support garments herein provide features that encourage patients to keep the device on for longer and/or uninterrupted periods of time, thereby increasing the quality of data collected about such arrhythmias. Additionally, features as described herein promote better patient compliance resulting in lower false positives and noise in the physiological signals collected from ECG electrodes and other sensors disposed within the support garment. For example, when patients wear the device for longer and/or uninterrupted periods of time, the device tracks cardiac events and distinguishes such events from noise over time.

The improvements incorporated in the support garment 50 can provide comfort and wearability to the patient by utilizing softer materials for at least some of the components of the support garment and by utilizing materials and construction features that are less likely to dig into and/or rub on the patient's skin in a painful or irritating manner.

In accordance with one or more examples, the support garment 50 is provided to keep the electrodes 11, 12 of an electrode assembly 25 associated with a wearable cardiac therapeutic device in place against the patient's body while remaining comfortable to wear. In particular, the electrode assembly 25 can include a plurality of ECG sensing electrodes 12 configured to sense ECG signals regarding a cardiac function of the patient and a plurality of therapy electrodes 11 configured to deliver transcutaneous defibrillation shocks or transcutaneous pacing pulses to the patient's heart. Examples of the wearable cardiac therapeutic devices in which the support garment 50 can be utilized include the wearable medical device 14 described above with reference to FIG. 10 and the wearable medical device 100 described in detail below with reference to FIGS. 14-16.

As shown in FIGS. 13A and 13B, in some examples, the support garment 50 is in the form of a vest or harness having a back portion 51 and sides extending around the front of the patient to form a belt 52. The ends 66, 67 of the belt 52 are connected at the front of the patient by a closure mechanism 65. The support garment 50 can further include straps as discussed in detail herein connecting the back portion 51 to the belt 52 at the front of the patient. The straps 53 have an adjustable size to provide a more customized fit to the patient. The straps 53 can also be selectively attached to the belt 22 at the front of the patient. The support garment 50 can be comprised of an elastic, low spring rate fabric material F that stretches appropriately to keep the electrodes 11, 12 in place against the patient's skin and is lightweight and breathable. The component materials of the fabric material F can be chosen for functionality, comfort, and biocompatibility. The component materials can be configured to wick perspiration from the skin. For example, the fabric material F can comprise a tricot fabric, the tricot fabric comprising nylon and spandex materials. The tricot fabric can comprise about 65% to about 90% nylon material, more particularly about 70% to about 85% nylon material, more particularly about 77% nylon material. It is to be appreciated that the fabric material F chosen for the support garment 50 can be comprised of any suitable materials or combinations of materials.

The support garment 50 as described in this disclosure can be configured for one-sided assembly of the electrode assembly 25 onto the support garment 50 such that the support garment 50 does not need to be flipped or turned over in order to properly position the therapy electrodes 11 and the sensing electrodes 12 on the support garment 50. The inside surface of the back portion 51 of the support garment 50 includes pocket(s) 56 for receiving one or two therapy electrodes 11 to hold the electrode(s) 11 in position against the patient's back. The pocket 56 is made from a non-clastic, conductive mesh fabric designed to isolate the metallic therapy electrode(s) 11 from the skin of the patient while allowing a conductive gel that can be automatically extruded from the electrode(s) 11 to easily pass through. The forces applied to the electrode(s) 11 by the fabric, in addition to the use of the conductive gel, can help ensure that proper contact and electrical conductivity with the patient's body are maintained, even during body motions. The fabric material of the pocket(s) 56 also maintains electrical contact between the electrode(s) 11 through the mesh material before the conductive gel is dispensed, which allows for monitoring of the therapy electrode(s) 11 to ensure that the electrode(s) 11 are positioned against the skin such that a warning can be provided by the wearable defibrillator 14 if the therapy electrode(s) 11 is not properly positioned. Another pocket 57 made from the same non-clastic, conductive mesh fabric is included on an inside surface of the belt 52 for receiving a therapy electrode 11 and holding the electrode 11 in position against the patient's left side. According to one example, the pockets 56, 57 are formed from an electrically conductive knit material. The material of the pockets 56, 57 can have a metal coating, such as a silver coating, applied thereto to provide electrical conductivity. The pockets 56, 57 can be closed by any suitable closure device 60, such as a hook and loop fastener.

The back portion 51 and the belt 52 of the support garment 50 can further incorporate attachment points 58 for supporting the sensing electrodes 12 in positions against the patient's skin in spaced locations around the circumference of the patient's chest. The attachment points 58 can include any of the features described herein with reference to FIG. 1A-3C, 7, and 9. The attachment points 58 can include hook-and-loop fasteners for attaching electrodes 12 having a corresponding fastener disposed thereon to the inside surface of the belt 52. The support garment 50 can further be provided with a flap 59 extending from the back portion 51. The flap 59 and the back portion 51 include a closure device 60 such as a hook and loop fastener for connecting the flap 59 to the inside surface of the back portion 51 in order to define a pouch or pocket for holding a distribution box 13 of the electrode assembly 25. The outer surface of the belt 52 can incorporate a schematic 30 (shown in FIG. 11) imprinted on the fabric for assisting the patient or medical professional in assembling the electrode assembly 25 onto the support garment 50.

Further discussion of the additional improvements incorporated into the support garment 50 for enhancing the patient's experience in wearing the support garment 50 for an extended period of time according to one or more examples of the present disclosure is provided below with reference to FIGS. 1A-8 and 13A-13B.

With reference to FIGS. 13A and 13B, according to an example of the present disclosure, the support garment 50 can be incorporated into a wearable cardiac therapeutic device with improved fasteners for fastening and supporting electrodes on the support garment 50.

The device includes a plurality of ECG sensing electrodes 12 configured to sense ECG signals regarding a cardiac function of the patient and the support garment 50 configured to support and hold the plurality of ECG sensing electrodes 12 against the patient's body. The device can further include a plurality of therapy electrodes 11 configured to deliver transcutaneous defibrillation shocks, transcutaneous cardioversion shocks, and/or transcutaneous pacing pulses to the patient's heart. The support garment 50 can be configured to support and hold the plurality of therapy electrodes 11 against the patient's body in accordance with implementations described herein. The support garment 50 includes a plurality of fasteners/attachment points 58 on an inside surface thereof for fastening and supporting the plurality of ECG sensing electrodes 12 on the support garment 50.

Each of the plurality of fasteners/attachment points 58 can include a hook and loop fastener patch affixed to a predetermined location on the inside surface of the support garment 50. Each of the plurality of ECG sensing electrodes 12 includes a corresponding hook and loop fastener patch configured to connect to a respective hook and loop fastener patch on the support garment 50.

The hook and loop fastener patches are configured to facilitate alignment and assembly of the respective ECG sensing electrodes 12 on the support garment 50 and to provide for fastening and support for the respective ECG sensing electrodes 12 on the support garment independent of the rotational orientation of the respective ECG sensing electrodes 12. This provides for easier assembly of the ECG sensing electrodes 12 on the support garment 50 and less error with respect to the assembly of the ECG sensing electrodes 12 on the support garment 50 resulting from misalignment of on the ECG sensing electrodes 12 with the hook and loop fastener patch of the fasteners/attachment points 58 on the support garment 50.

According to an example, each of the hook and loop fastener patches has a length of about 0.5″ to about 3.0″ to about and a width of about 0.5″ to about 3.0″. According to another example, each of the circular hook-and-loop fastener patches has a length and width of about 1.25″, respectively. It is to be appreciated that the hook and loop fastener patches can be of any suitable size.

According to an example, the hook and loop fastener patch can comprise a nylon, polyester, or polypropylene material. It is to be appreciated that the hook and loop fastener patch can comprise any suitable materials.

According to an example, the hook and loop fastener patch are permanently affixed to the interior surface of the support garment 50 by sewing. It is to be appreciated that the hook and loop fastener patches can be affixed to the support garment 50 by any suitable technique.

With reference to FIGS. 10, 13A, and 13B, according to an example of the present disclosure, the support garment 50 can be incorporated into a wearable cardiac therapeutic device with improved features for assembly of therapy electrodes 11 on the support garment 50.

The device includes a plurality of therapy electrodes 11 configured to deliver transcutaneous defibrillation shocks or transcutaneous pacing pulses to a patient's heart and the support garment 50 configured to support and hold the plurality of therapy electrodes 11 against the patient's body. The device can further include a plurality of ECG sensing electrodes 12 configured to sense ECG signals regarding a cardiac function of the patient. The support garment 50 can be configured to support and hold the plurality of ECG sensing electrodes 12 against the patient's body.

The support garment 50 includes a plurality of support pockets 56, 57 disposed on an inside surface of the support garment 50 for supporting the plurality of therapy electrodes 11 on the support garment 50 and a plurality of corresponding closure devices 61, such as a hook and loop fastener or other suitable closure devices. At least one closure device 61 is fastened to each of the plurality of support pockets 56, 57. The closure devices 61 are configured to facilitate opening and closing of the plurality of support pockets 56, 57 for assembly of the plurality of therapy electrodes 11 therein. It is to be appreciated that the closure device(s) 61 can be fastened to the support pockets 56, 57 in any suitable manner.

Aspects of the present disclosure are directed to monitoring and/or therapeutic medical devices configured to identify a patient physiological event and, in response to the identified event, to provide a notification to the patient wearing the device. The notification can include an instruction or request to perform a patient response activity. Successful completion of the patient response activity can cause the device to suspend or delay a device function, such as administering a treatment to a patient and/or issuing an alert or alarm.

In some examples, the medical device includes monitoring circuitry configured to sense physiological information of a patient. The controller can be configured to detect the patient physiological event based, at least in part, on the sensed physiological information. A patient event can be a temporary physiological problem or abnormality, which can be representative of an underlying patient condition. A patient event can also include injuries and other non-recurring problems that are not representative of underlying physiological condition of the patient. A non-exhaustive list of patient events that can be detected by an external medical device includes, for example: bradycardia, ventricular tachycardia (VT) or ventricular fibrillation (VF), atrial arrhythmias such as premature atrial contractions (PACs), multifocal atrial tachycardia, atrial flutter, and atrial fibrillation, supraventricular tachycardia (SVT), junctional arrhythmias, tachycardia, junctional rhythm, junctional tachycardia, premature junctional contraction, and ventricular arrhythmias such as premature ventricular contractions (PVCs) and accelerated idioventricular rhythm.

In some examples, the device controller is configured to notify the patient of the detection of the one or more events and to receive a patient response to the notification. The patient response can include performing a response activity identifiable by an input component associated with the medical device. In general, the response activity is selected to demonstrate or to provide information about the status of the patient and, in particular, to confirm that the patient is conscious and substantially aware of his or her surroundings. The response activity or activities can also be configured to confirm patient identity (e.g., that the person providing the response is the patient, rather than a bystander or impostor). The response activity can also demonstrate or test a patient ability such as one or more of psychomotor ability, cognitive awareness, and athletic/movement ability. In some examples, the response activity can be a relatively simple action, such as making a simple or reflexive movement in response to a stimulus applied by the device. In other examples, more complex activities, such as providing answers to questions requiring reasoning and logical analysis can be required. The device can be configured to select a particular response activity based on characteristics of the patient and/or the detected patient event.

In some examples, the device can instruct the patient to perform several actions that are each representative of patient ability. In other modes, the device can instruct the patient to perform different types of activities that are representative of different patient abilities. For example, the device can instruct the patient to perform a single activity requiring several patient abilities to complete correctly. Alternatively, the device can instruct the patient to perform a first activity representative of a first patient ability and, once the first activity is correctly completed, to perform a second activity representative of a second patient ability.

This disclosure relates to components, modules, subsystems, circuitry, and/or techniques for use in external medical devices. For example, such components, modules, subsystems, circuitry, and/or techniques can be used in the context of medical devices for providing treatment to and/or monitoring a patient. For example, such medical devices can include monitoring devices configured to monitor a patient to identify occurrence of certain patient events. In some implementations, such devices are capable, in addition to monitoring for patient conditions, of providing treatment to a patient based on detecting a predetermined patient condition.

In some examples, the medical device can be a patient monitoring device, which can be configured to monitor one or more of a patient's physiological parameters without an accompanying treatment component. For example, a patient monitor can include a cardiac monitor for monitoring a patient's cardiac information. Such cardiac information can include, without limitation, heart rate, ECG data, heart sounds data from an acoustic sensor, and other cardiac data. In addition to cardiac monitoring, the patient monitor can perform monitoring of other relevant patient parameters, including glucose levels, blood oxygen levels, lung fluids, lung sounds, and blood pressure.

FIGS. 14-16 illustrate an exemplary wearable medical device 100, such as a wearable defibrillator, which can incorporate the exemplary features of the support garment described in this disclosure. The wearable medical device 100 includes a plurality of sensing electrodes 112 that can be disposed at various positions about the patient's body. The sensing electrodes 112 are electrically coupled to a medical device controller 120 through a connection pod 130. In some implementations, some of the components of the wearable medical device 100 are affixed to a garment 110 that can be worn on the patient's torso. According to an example of the present disclosure, the garment 110 shown in FIG. 14 can be the same as the support garment 50 discussed above with reference to FIG. 13A-13B.

The devices described herein are capable of continuous, substantially continuous, long-term and/or extended use or wear by, or attachment or connection to, a patient. In this regard, the device can be configured to be used or worn by, or attached or connected to, a patient, without substantial interruption, for example, up to hours or beyond (e.g., weeks, months, or even years). For example, in some implementations, such a period of use or wear can be at least 4 hours. For example, such a period of use or wear can be at least 24 hours or one day. For example, such a period of use or wear can be at least 7 days. For example, such a period of use or wear can be at least one month. In some implementations, such devices can be removed for a period of time before use, wear, attachment, or connection to the patient is resumed, e.g., to change batteries, to change or wash the garment, and/or to take a shower. Similarly, the device can be configured for continuous, substantially continuous, long-term and/or extended monitoring of one or more patient physiological conditions. For instance, in addition to cardiac monitoring, the medical device can be capable of monitoring a patient for other physiological conditions. Accordingly, in implementations, the device can be configured to monitor blood oxygen, temperature, glucose levels, sleep apnea, snoring and/or other sleep conditions, heart sounds, lung sounds, tissue fluids, etc. using a variety of sensors including radio frequency (RF) sensors, ultrasonic sensors, electrodes, etc. In some instances, the device can carry out its monitoring in periodic or aperiodic time intervals or times. For example, the monitoring during intervals or times can be triggered by a patient action or another event. For example, one or more durations between periodic or aperiodic intervals or times can be patient and/or other non-patient user configurable.

For example, as shown in FIG. 14, the controller 120 can be mounted on a belt worn by the patient. The sensing electrodes 112 and connection pod 130 can be assembled or integrated into the garment 110 as shown. The sensing electrodes 112 are configured to monitor the cardiac function of the patient (e.g., by monitoring one or more cardiac signals of the patient). While FIG. 14 shows four sensing electrodes 112, additional sensing electrodes can be provided, and the plurality of sensing electrodes 112 can be disposed at various locations about the patient's body.

The wearable medical device 100 can also optionally include a plurality of therapy electrodes 114 that are electrically coupled to the medical device controller 120 through the connection pod 130. The therapy electrodes 114 are configured to deliver one or more therapeutic transcutaneous defibrillating shocks, transcutaneous pacing pulses, and/or TENS pulses to the body of the patient if it is determined that such treatment is warranted. The connection pod 130 can include electronic circuitry and one or more sensors (e.g., a motion sensor, an accelerometer, etc.) that are configured to monitor patient activity. In some implementations, the wearable medical device 100 can be a monitoring-only device that omits the therapy delivery capabilities and associated components (e.g., the therapy electrodes 114). In some implementations, various treatment components can be packaged into various modules that can be attached or removed from the wearable medical device 100 as needed. As shown in FIG. 14, the wearable medical device 100 can include a patient interface pod 140 that is electrically coupled to, integrated in, and/or integrated with the patient interface of the medical device controller 120. For example, the patient interface pod 140 can include patient interface elements such as a speaker, a microphone responsive to patient input, a display, an interactive touch screen responsive to patient input, and/or physical buttons for input.

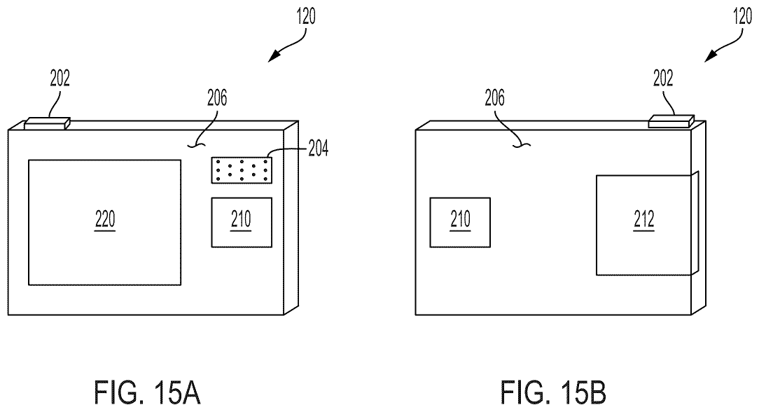

With reference to FIGS. 15A and 15B, an example of the medical device controller 120 is illustrated. The controller 120 can be powered by a rechargeable battery 212. The rechargeable battery 212 can be removable from a housing 206 of the medical device controller 120 to enable a patient and/or caregiver to swap a depleted (or near-depleted) battery 212 for a charged battery. The controller 120 includes a patient interface such as a touch screen 220 that can provide information to the patient, caregiver, and/or bystanders. In some implementations, in addition to or instead of a touch screen 220, the controller 120 can interact with the patient (e.g., receive patient input or provide information to the patient as described herein) via patient interface pod 140 (shown in FIG. 14). The patient interface pod 140 can be operatively coupled to the controller 120. In an example, the controller 120 can be configured to detect that if the patient interface pod 140 is operatively coupled to the controller 120, the controller 120 can then disable the patient interface elements of the controller 120 (e.g., touch screen 220) and instead communicate via the patient interface pod 140. The patient interface pod 140 can be wirelessly coupled with the controller 120. The patient interface pod 140 can take other forms and include additional functionality. For instance, the patient interface pod 140 can be implemented on a smartphone, tablet, or other mobile device carried by the patient. In another example, the patient interface pod 140 can be worn as a watch about the wrist of the patient, or as a band about an upper arm of the patient. In some implementations, the controller 120 can communicate certain alerts and information and/or be responsive to patient input via both the patient interface elements included in the controller 120 and the patient interface pod 140. The patient and/or caregiver can interact with the touch screen 220 or the patient interface pod 140 to control the medical device 100. The controller 120 also includes a speaker 204 for communicating information to the patient, caregiver, and/or the bystander. The controller 120 (and/or the patient interface pod 140) can include one or more response buttons 210. In some examples, when the controller 120 determines that the patient is experiencing cardiac arrhythmia, the speaker 204 can issue an audible alarm to alert the patient and bystanders to the patient's medical condition. In some examples, the controller 120 can instruct the patient to press one or both of the response buttons 210 to indicate that he or she is conscious, thereby instructing the medical device controller 120 to withhold the delivery of therapeutic defibrillating shocks. If the patient does not respond to an instruction from the controller 120, the medical device 100 can determine that the patient is unconscious and proceed with the treatment sequence, culminating in the delivery of one or more defibrillating shocks to the body of the patient. In some examples, as discussed in further detail herein, the controller 120 can additionally or alternatively instruct the patient to perform a response activity to indicate that he or she is conscious and further provide information to the controller 120 regarding the patient's status. For example, the controller 120 can instruct the patient to touch or manipulate the touch screen 220 or an interactive display on the patient interface pod 140 in a coordinated manner to confirm that he or she is conscious and has requisite awareness and/or psychomotor ability. In this way, the patient response confirms not only that buttons 210 were pressed, but that the patient is sufficiently conscious and aware to perform a response activity as instructed. The medical device controller 120 can further include a port 202 to removably connect sensing devices (e.g., ECG sensing electrodes 112) and/or therapeutic devices (e.g., therapy electrodes 114 shown in FIG. 19) to the medical device controller 120.

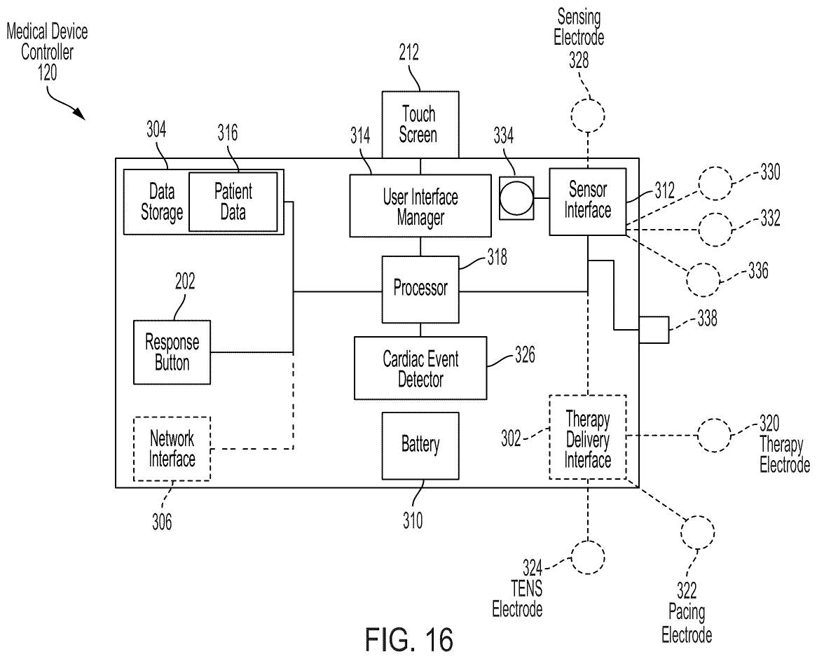

With reference to FIG. 16, a schematic example of the medical device controller 120 of FIGS. 14, 15A, and 15B is illustrated. As shown in FIG. 16, the controller 120 includes at least one processor 318, a patient interface manager 314, a sensor interface 312, an optional therapy delivery interface 302, data storage 304 (which can include patient data storage 316), an optional network interface 306, a patient interface 308 (e.g., including the touch screen 220 shown in FIGS. 21A and 21B), and a battery 310. The sensor interface 312 can be coupled to any one or combination of sensors to receive information indicative of cardiac activity. For example, the sensor interface 312 can be coupled to one or more sensing devices including, for example, sensing electrodes 328, contact sensors 330, pressure sensors 332, accelerometers or motion sensors 334, and radio frequency (RF)-energy based sensors 331 (e.g., tissue fluid sensors). The controller 120 can also include an optical sensor 336, such as a digital camera, for capturing static or video images of the device surroundings. Although designs from different vendors are different, a digital camera usually consists of a charge-coupled device (CCD) or complementary metal-oxide-semiconductor (CMOS) imaging sensor, a lens, a multifunctional video control chip, and a set of discrete components (e.g., capacitor, resistors, and connectors). The therapy delivery interface 302 (if included) can be coupled to one or more electrodes that provide therapy to the patient including, for example, one or more therapy electrodes 320, pacing electrodes 322, and/or TENS electrodes 324. The sensor interface 312 and the therapy delivery interface 302 can implement a variety of coupling and communication techniques for facilitating the exchange of data between the sensors and/or therapy delivery devices and the controller 120.

The medical device controller 120 can comprise one or more input components configured to receive a response input from the patient. The input components can comprise at least one of: the response button 210; the touch screen 220; an audio detection device, such as a microphone 338; the motion sensor 334; the contact sensor 330; the pressure sensor 332; a gesture recognitions component, such as the optical sensor 336; or a patient physiological sensor, such as the sensing electrodes 328.

In some examples, the medical device controller 120 includes a cardiac event detector 326 to monitor the cardiac activity of the patient and identify cardiac events experienced by the patient based on received cardiac signals. In other examples, cardiac event detection can be performed using algorithms for analyzing patient ECG signals obtained from the sensing electrodes 328. Additionally, the cardiac event detector 326 can access patient templates (e.g., which can be stored in the data storage 304 as patient data 316) that can assist the cardiac event detector 326 in identifying cardiac events experienced by the particular patient (e.g., by performing template matching algorithms).

The at least one processor 318 can perform a series of instructions that control the operation of the other components of the controller 120. In some examples, the patient interface manager 314 is implemented as a software component that is stored in the data storage 304 and executed by the at least one processor 318 to control, for example, the patient interface component 308. The patient interface manager 314 can control various output components and/or devices of the medical device controller 300 (e.g., patient interface 220 and/or patient interface pod 140 shown in FIG. 14) to communicate with external entities consistent with various acts and/or display screens described herein. For example, such output components and/or devices can include speakers, tactile and/or vibration output elements, visual indicators, monitors, displays, LCD screens, LEDs, Braille output elements, and the like. Additionally, the patient interface manager 314 can be integrated with the treatment-providing components of the controller 120 so that the patient can control and, in some cases, suspend, delay, or cancel treatment using the patient interface.

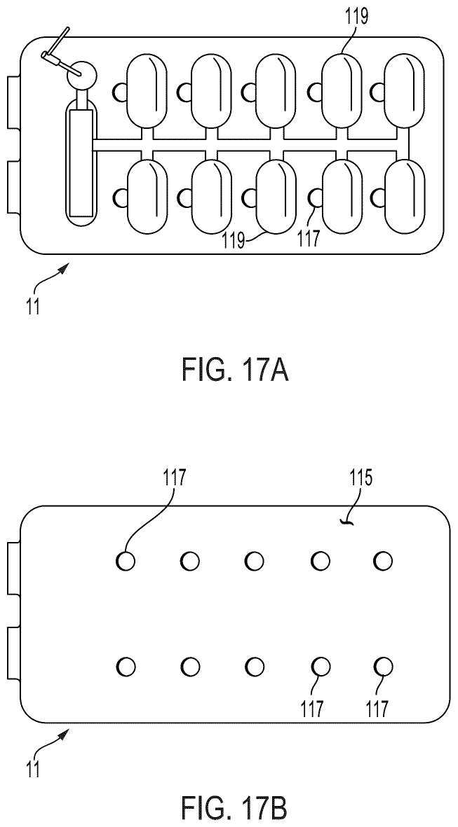

FIGS. 17A and 17B illustrate the therapy electrode 11 utilized in the various examples described herein. The therapy electrode 11 includes a conductive bottom surface 115 configured to establish an electrical interface with the patient's skin through the pocket 57. The conductive bottom surface 115 can be metallic. The conductive bottom surface 115 can include one or more apertures 117 through which a conductive gel can be dispensed to improve the electrical interface between the conductive bottom surface 115 and the patient's skin. The conductive gel can be dispensed from one or more gel packs 119 arranged in fluid communication with the apertures 117 on a side of the conductive bottom surface 115 facing away from the patient. The controller 120 (shown in FIG. 16) can control dispensing of the conductive gel from the gel packs 119.

Although a wearable medical device and a support garment for such a device have been described in detail for the purpose of illustration based on what is currently considered to be the most practical examples, it is to be understood that such detail is solely for that purpose and that the subject matter of this disclosure is not limited to the disclosed examples, but, on the contrary, is intended to cover modifications and equivalent arrangements. For example, it is to be understood that this disclosure contemplates that, to the extent possible, one or more features of any example can be combined with one or more features of any other example.

1. A non-invasive wearable ambulatory cardiac defibrillator comprising:
a garment configured to be worn around a torso of a patient;
at least one sensing electrode attached to the garment and configured to sense electrical signal(s) at the surface of the patient's skin indicative of electrical activity of the patient's heart;
at least one therapy electrode attached to the garment and configured to deliver one or more defibrillation pulses to the patient; and
a controller in communication with the at least one sensing electrode and the therapy electrodes, the controller configured to receive the electrical signal(s) from the at least one sensing electrode and to cause delivery of the one or more defibrillation pulses from the at least one therapy electrode based on the controller detecting a cardiac arrhythmia in the received electrical signal(s);
wherein the garment comprises:
a main garment portion configured to engage the torso of the patient;
an isolation zone material disposed within the main garment portion and to which one of the sensing electrodes is attached; and
at least one movement absorption region connecting the main garment portion to the isolation zone material, the at least one movement absorption region having a different elasticity than the main garment portion and the isolation zone material.

2. The defibrillator of claim 1, wherein the at least one movement absorption region comprises at least one of
a mesh having a less dense knit structure than at least one of the main garment portion and the isolation zone material;
a different thickness than at least one of the main garment portion and the isolation zone material; or
a different stiffness than at least one of the main garment portion and the isolation zone material.

3-4. (canceled)

5. The defibrillator of claim 1, wherein the garment further comprises a plurality of connecting portions extending between the main garment portion and the isolation zone material.

6. The defibrillator of claim 1, wherein the at least one movement absorption region comprises a plurality of movement absorption regions arranged around a perimeter of the isolation zone material.

7. The defibrillator of claim 6, wherein each of the plurality of movement absorption regions is arcuate.

8. The defibrillator of claim 1, wherein the isolation zone material is rectangular or circular.

9. (canceled)

10. The defibrillator of claim 1, wherein the isolation zone material comprises a reinforcing support film to which the one of the sensing electrodes is attached.

11. The defibrillator of claim 1, wherein the at least one movement absorption region is configured to induce a normal force on the one of the sensing electrodes ranging from about 0.1 psi to about 0.6 psi.

12. The defibrillator of claim 1, wherein the at least one movement absorption region is configured to induce a normal force on the one of the sensing electrodes ranging from about 0.3 psi to about 0.6 psi.

13. (canceled)

14. The defibrillator of claim 1, wherein the garment further comprises a plurality of isolation zones, wherein each of the at least one sensing electrode is attached to one of the plurality of isolation zone materials.

15. The defibrillator of claim 1, wherein the controller is configured generate ECG information from the electrical signal(s) received from the at least one sensing electrode and to cause delivery of the one or more therapeutic pulses from the at least one therapy electrode.

16. A non-invasive wearable ambulatory cardiac defibrillator, comprising:
a garment configured to be worn around a torso of a patient;
at least one sensing electrode attached to the garment and configured to sense electrical signal(s) at the surface of the patient's skin indicative of electrical activity of the patient's heart;
at least one therapy electrode attached to the garment and configured to deliver one or more defibrillation pulses to the patient; and
a controller in communication with the at least one sensing electrode and the therapy electrodes, the controller configured to receive the electrical signal(s) from the at least one sensing electrode and to cause delivery of the one or more defibrillation pulses from the at least one therapy electrode based on the controller detecting a cardiac arrhythmia in the received electrical signal(s);
wherein the garment comprises:
a belt portion configured to wrap around the torso or waist of the patient; and
at least one insert disposed in the belt portion, the at least one insert configured to have a greater elasticity in a circumferential direction than an adjacent section of the belt portion.

17. The defibrillator of claim 16, wherein the belt portion is adjustable between a minimum circumference and a maximum circumference, and
wherein a location of the at least one sensing electrode about a circumference of the belt portion is different at the minimum circumference than at the maximum circumference due to stretching of the at least one insert.

18. The defibrillator of claim 17, wherein a location of a first of the at least one electrodes changes by a first distance when the belt portion is adjusted from the minimum circumference to the maximum circumference;
wherein a location of a second of the at least one electrodes changes by a second distance when the belt portion is adjusted from the minimum circumference to the maximum circumference, and
wherein the first distance is different than the second distance.

19. The defibrillator of claim 16, wherein the at least one insert comprises a plurality of inserts.

20. The defibrillator of claim 19, wherein the plurality of inserts are configured with at least one of
each of the plurality of inserts is-disposed between two of the sensing electrodes to allow a distance between the two sensing electrodes to be adjusted by stretching the insert;
the plurality of inserts disposed asymmetrically about a circumference of the belt portion; or
an elasticity of at least one of the plurality of the inserts being different than an elasticity of at least one other of the plurality of insert.

21-22. (canceled)

23. The defibrillator of claim 16, wherein the controller is configured generate ECG information from the electrical signal(s) received from the at least one sensing electrode and to cause delivery of the one or more therapeutic pulses from the at least one therapy electrode.

24. A non-invasive wearable ambulatory cardiac defibrillator, comprising:
a garment configured to be worn around a torso of a patient;
at least one sensing electrode attached to the garment and configured to sense electrical signal(s) at the surface of the patient's skin indicative of electrical activity of the patient's heart;
at least one therapy electrode attached to the garment and configured to deliver one or more defibrillation pulses to the patient; and
a controller in communication with the at least one sensing electrode and the therapy electrodes, the controller configured to receive the electrical signal(s) from the at least one sensing electrode and to cause delivery of the one or more defibrillation pulses from the at least one therapy electrode based on the controller detecting a cardiac arrhythmia in the received electrical signal(s),
wherein the garment comprises at least one graduated thickness section to which the at least one sensing electrode is attached, the at least one graduated thickness section having a varying thickness, the at least one graduated thickness section configured to apply a predetermined normal force to the at least one sensing electrode.

25. The defibrillator of claim 24, wherein the at least one graduated thickness section comprises a depression into which the at least one sensing electrode is at least partially recessed.

26. (canceled)

27. The defibrillator of claim 24, wherein the varying thickness of the graduated thickness section comprises at least one of
tapering from a minimum thickness at an outer edge of the graduated thickness section to a maximum thickness at an inner region of the graduated thickness section;
tapering non-linearly; or
a constant thickness section where the at least one sensing electrode is attached to the graduated thickness section.

28-33. (canceled)
